(19) United States
(12) Patent Application Publication
(10) Pub. No.: US 2021/0196648 A1
(43) Pub. Date: Jul. 1, 2021

(54) DRUG-CONTAINING MULTILAYER FILM AND METHOD FOR FORMING THE SAME

(71) Applicant: Industrial Technology Research Institute, Hsinchu (TW)

(72) Inventors: Hsin-Hsin SHEN, Zhudong Township (TW); Yuchi WANG, New Taipei City (TW); Li-Hsin LIN, Zhubei City (TW); Ming-Chia YANG, Taipei City (TW); Hsiu-Hua HUANG, Hsinchu City (TW); Liang-Cheng SU, Kaohsiung City (TW); Ying-Hsueh CHAO, Tainan City (TW); Jing-En HUANG, Hsinchu City (TW)

(73) Assignee: Industrial Technology Research Institute, Hsinchu (TW)

(21) Appl. No.: 16/731,979

(22) Filed: Dec. 31, 2019

(51) Int. Cl. A61K 9/70

(52) U.S. Cl. CPC A61K 9/7023

(57) ABSTRACT

A drug-containing multilayer film provided. The drug-containing multilayer film includes: a drug-containing layer; and an anti-adhesion layer on a surface of the drug-containing layer. The drug-containing layer is composed of a first composition including a first polymer material and a drug, and the first polymer material includes at least one selected from the group consisting of: polylactic acid (PLA) and polyethylene glycol (PEG), and the weight ratio of the first polymer material to the drug is about 1:0.01-0.3. The anti-adhesion layer is composed of a second composition, and the second composition includes a second polymer material, and the second polymer material includes at least one selected from the group consisting of polylactic acid and polyethylene glycol.

### TECHNICAL FIELD

The present disclosure is related to a film for medical use, and is particularly related to a drug-containing multilayer film and a method for preparing the same.

### BACKGROUND

Based on research, adhesions after surgery can cause a variety of complications, such as: small intestinal obstructions, chronic pelvic pain, infertility, etc. For this reason, the prevention of adhesion after surgery can not only reduce the patient's subsequent discomfort and decrease the risk of having to undergo reoperation due to adhesion problems, but it can also reduce medical expenses.

In addition to surgery causing adhesions, there may be pain or infection during wound repair after surgery. Therefore, drugs such as analgesics and antibiotics are needed to reduce the patient's discomfort.

At present, there are four main methods of postoperative analgesia, intramuscular injection, intravenous injection, patient-controlled analgesia, and continuous epidural analgesia. Both intramuscular injection and intravenous injection require repeated administration of the drug within a few hours to maintain the analgesic effect, but they can also have side effects.

Therefore, there is an urgent need for medical equipment that can simultaneously reduce or avoid the adhesion and discomfort caused by surgery.

### SUMMARY

The present disclosure provides a drug-containing multilayer film comprising: a drug-containing layer; and an anti-adhesion layer over a surface of the drug-containing layer. The drug-containing layer is composed of a first composition which comprises a first polymer material and a drug, and the first polymer material comprises at least one selected from the group consisting of: polylactic acid (PLA) and polyethylene glycol (PEG), and a weight ratio of the first polymer material to the drug is 1:0.01-0.3. The anti-adhesion layer is composed of a second composition which comprises a second polymer material, and the second polymer material comprises at least one selected from the group consisting of: polylactic acid and polyethylene glycol.

The present disclosure also provides a method for forming a drug-containing multilayer film, comprising the following manner (a) or (b). The manner (a) comprises: (i) drying a first solution to form a film to form a drug-containing layer; and (ii) drying a second solution to form a film over a surface of the drug-containing layer to form an anti-adhesion layer. The manner (b) comprises (i′) drying a second solution to form a film to form an anti-adhesion layer; and (ii′) drying a first solution to form a film over the anti-adhesion layer to form a drug-containing layer, wherein a solute of the first solution comprises a first polymer material and a drug, and the first polymer material comprises at least one selected from the group consisting of: polylactic acid and polyethylene glycol, and a content of the first polymer material in the first solution is about 5-30 wt %, and a weight ratio of the first polymer material to the drug is 1:0.01-0.3. A solute of the second solution comprises a second polymer material, and the second polymer material comprises at least one selected from the group consisting of: polylactic acid and polyethylene glycol, and a content of the second polymer material in the second solution is about 5-30 wt %.

A detailed description is given in the following embodiments with reference to the accompanying drawings.

### BRIEF DESCRIPTION OF DRAWINGS

The present invention can be more fully understood by reading the subsequent detailed description and examples with references made to the accompanying drawings, wherein:

FIG. 1 shows one embodiment of a method for forming a drug-containing multilayer film of the present disclosure;

FIG. 2 shows another embodiment of a method for forming a drug-containing multilayer film of the present disclosure;

FIG. 3 shows, in one embodiment, a film-forming method for a drug-containing multilayer film of the present disclosure;

FIG. 4 shows yet another embodiment of a method for forming a drug-containing multilayer film of the present disclosure;

FIG. 5 shows yet another embodiment of a method for forming a drug-containing multilayer film of the present disclosure;

FIG. 6A shows photographs of a cell assay in an anti-adhesion test for the anti-adhesion film;

FIG. 6B shows results of the cell viability analysis of the cell experiment in the anti-adhesion test for the anti-adhesion film;

FIG. 7 shows a surgical method of an animal experiment in an anti-adhesion test for the anti-adhesion film;

FIG. 8A shows photographs of the surgical area of each group of rats in an animal experiment in the anti-adhesion test for the anti-adhesion films;

FIG. 8B shows the percentages of rats with mild adhesion and moderate or severe adhesion in each group of animal experiments in an anti-adhesion test of the anti-adhesion films;

FIG. 9 shows a photograph of the drug-containing multilayer film RM disclosed in this disclosure;

FIG. 10 shows the results of the immediate release test for the drug-containing multilayer films M-01, M-02, M-03, M-04, M-05 and M-06 of the present disclosure;

FIG. 11 shows the results of the immediate release test for the drug-containing multilayer film M-02 (without protective layer) and the drug-containing multilayer films M-07, M-08, M-09, and M-10 (with a protective layer) of the present disclosure;

FIG. 12 shows results of the release test for the drug-containing multilayer film M-05 (without protective layer) and the drug-containing multilayer films M-11 and M-12 of the present disclosure;

FIG. 13 shows the release test results for the drug-containing multilayer film M-11 (the drug-containing layer has no microstructure) and the drug-containing multilayer films MS-1, MS-2, and MS-3 of the present disclosure;

FIG. 14 shows the results of scanning electron microscope analysis for the drug-containing multilayer films MS-1, MS-2, and MS-3 of the present disclosure;

FIG. 15 shows the results of release tests on the drug-containing multilayer films MS-1, MS-2, MS-1-B and MS-2-B; and

FIG. 16 shows the results of scanning electron microscope analysis for the drug-containing multilayer films MS-1-B and MS-2-B.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

The present disclosure provides a drug-containing multilayer film. The drug-containing multilayer film of the present disclosure is a biodegradable non-fiber form film, and can be well attached on a surgical wound or a diffuse wound without needing sutures or another fixing manner, and can prevent leakage of tissue fluid. Furthermore, the drug-containing multilayer film of the present disclosure has an anti-adhesion effect to prevent the occurrence of unwanted tissue adhesion, and at the same time can release the drug needed to a wound or tissue area to which the film is attached.

The drug-containing multilayer film of the present disclosure mentioned above may comprise, but is not limited to, a drug-containing layer and an anti-adhesion layer over a surface of the drug-containing layer.

The foregoing drug-containing layer may be composed of a first composition. The first composition mentioned above may comprise a first polymer material and a drug, but it is not limited thereto. In one embodiment, the weight ratio of the first polymer material to the drug may be about 1:0.01-0.3, such as 1:0.05-0.25, about 1:0.1, about 1:0.15, about 1:0.2, but it is not limited thereto.

Examples of the first polymer material mentioned above may comprise, but are not limited to, polylactic acid (PLA), polyethylene glycol (PEG) or any combination thereof.

Examples of the polylactic acid in the first polymer material mentioned above may comprise polylactic acid with different viscosities and any combination thereof. For instance, the polylactic acid in the examples of the first polymer material mentioned above may comprise a first polylactic acid, a second polylactic acid or a combination thereof, wherein the first polylactic acid and the second polylactic acid may have different viscosities. The viscosity of the first polylactic acid may be about 0.05-1 dl/g, such as about 0.4-0.6 dl/g, but it is not limited thereto, while the viscosity of the second polylactic acid may be about 1.1-3.0 dl/g, such as about 1.6-2.4 dl/g, but it is also not limited thereto.

Moreover, the polyethylene glycol in the first polymer material mentioned above may comprise polyethylene glycol with different average molecular weight and any combination thereof. For instance, the polyethylene glycol in the examples of the first polymer material mentioned above may comprise a first polyethylene glycol, a second polyethylene glycol or a combination thereof, wherein the first polyethylene glycol and the second polyethylene glycol may have different average molecular weights. The average molecular weight of the first polyethylene glycol may be about 100-1,000, such as about 285-315, but it is not limited thereto while the average molecular weight of the second polyethylene glycol may be about 4,000-10,000, as about 5,000-7,000, but it is also not limited thereto.

In one embodiment, in the foregoing drug-containing layer, the first polymer material comprises polylactic acid. In this embodiment, the polylactic acid may be the first polylactic acid, the second polylactic acid or the combination thereof mentioned above.

In another embodiment, in the foregoing drug-containing layer, the first polymer material may comprise both the polylactic acid and the polyethylene glycol. In this embodiment, the polylactic acid may be the foregoing first polylactic acid or the foregoing second polylactic acid, and the polyethylene glycol may be the foregoing first polyethylene glycol, the foregoing second polyethylene glycol or a combination thereof. In this embodiment, in the foregoing drug-containing layer, the weight ratio of the polylactic acid to the polyethylene glycol may be about 1:0.05-1, such as about 1:0.1, about 1:0.2, about 1:0.25, about 1:0.3, about 1:0.4, about 1:0.43, about 1:0.5, about 1:0.75, but it is not limited thereto.

Furthermore, in one specific embodiment, the first polymer material may comprise both the polylactic acid and the polyethylene glycol, and the polylactic acid may be the foregoing first polylactic acid or the foregoing second polylactic acid, and the polyethylene glycol may be the foregoing first polyethylene glycol or the foregoing second polyethylene glycol.

In another specific embodiment, the first polymer material may comprise both the polylactic acid and the polyethylene glycol, and the polylactic acid may be the foregoing first polylactic acid or the foregoing second polylactic acid, and the polyethylene glycol may be a combination of the foregoing first polyethylene glycol and the foregoing second polyethylene glycol. In this specific embodiment, the weight ratio of the foregoing first polyethylene glycol to the foregoing second polyethylene glycol may be about 1:0.05-20, such as 1:0.1, 1:0.2, 1:0.5, 1:1, 1:5, 1:10, 1:15, but it is not limited thereto.

In addition, the drug in the drug-containing layer suitable for the drug-containing multilayer film of the present disclosure is not particularly limited, and can be selected according to practical need, as long as it can be released through the drug-containing multilayer film of the present disclosure, and does not generate an adverse effect together with other ingredients in the drug-containing layer, such as affecting the formation of the drug-containing layer or inactivating other drug, etc.

Examples of the drug in the drug-containing layer may comprise an analgesic, an anti-inflammatory and analgesic drug, an antibiotic, but they are not limited thereto. The analgesic may comprise, but is not limited to, lidocaine hydrochloride, morphine hydrochloride, fentanyl, etc. The anti-inflammatory and analgesic drug may comprise, but is not limited to, nonsteroidal anti-inflammatory drug (NSAID), such as ibuprofen, naproxen, aspirin, fenbufen, indometacin, diclofenac sodium. The antibiotic may comprise, but is not limited to, vancomycin, rifampin, etc.

In one specific embodiment, in the drug-containing multilayer film of the present disclosure the first polymer material in the drug-containing layer is the polylactic acid, and the polylactic acid is the first polylactic acid of which the viscosity may be about 0.05-1 dl/g, and the drug in the drug-containing layer may comprise an analgesic, an anti-inflammatory and analgesic drug or a combination thereof. Furthermore, in this specific embodiment, the drug-containing multilayer film of the present disclosure has an effect of immediate release of the drug.

In another specific embodiment, in the drug-containing multilayer film of the present disclosure, the first polymer material in the drug-containing layer is the polylactic acid, and the polylactic acid is the second polylactic acid of which the viscosity may be about 1.1-3.0 dl/g, and the drug in the drug-containing layer may comprise an analgesic, an antibiotic or a combination thereof. Furthermore, in this specific embodiment, the drug-containing multilayer film has an effect of sustained release of the drug.

In another specific embodiment, in the drug-containing multilayer film of the present disclosure, the first polymer material in the drug-containing layer is a combination of polylactic acid and polyethylene glycol, and the polylactic acid is the first polylactic acid of which the viscosity may be about 0.05-1 dl/g, and the polyethylene glycol is the first polyethylene glycol of which the average molecular weight may be about 100-1,000 mentioned above or the second polyethylene glycol of which the average molecular weight may be about 4,000-10,000 mentioned above, and the drug in the drug-containing layer may comprise an analgesic, an anti-inflammatory and analgesic drug or a combination thereof. Furthermore, in this specific embodiment, the drug-containing multilayer film of the present disclosure has an effect of immediate release of the drug.

In another specific embodiment, in the drug-containing multilayer film of the present disclosure, the first polymer material in the drug-containing layer is a combination of polylactic acid and polyethylene glycol, and the polylactic acid is the second polylactic acid of which the viscosity may be about 1.1-3.0 dl/g, and the polyethylene glycol is the first polyethylene glycol of which the average molecular weight may be about 100-1,000 mentioned above or the second polyethylene glycol of which the average molecular weight may be about 4,000-10,000 mentioned above, and the drug in the drug-containing layer may comprise an analgesic, an antibiotic or a combination thereof. Moreover, in this specific embodiment, the drug-containing multilayer film has an effect of sustained release of the drug.

In another specific embodiment, in the drug-containing multilayer film of the present disclosure, the first polymer material in the drug-containing layer is a combination of polylactic acid and polyethylene glycol, and the polylactic acid is the first polylactic acid of which the viscosity may be about 0.05-1 dl/g, and the polyethylene glycol is a combination of the first polyethylene glycol of which the average molecular weight may be about 100-1,000 mentioned above and the second polyethylene glycol of which the average molecular weight is about 4,000-10,000 mentioned above, and the weight ratio of the first polyethylene glycol to the second polyethylene glycol is about 1:0.01-10. The drug in the drug-containing layer may comprise an analgesic, an anti-inflammatory and analgesic drug or a combination thereof. Furthermore, in this specific embodiment, the drug-containing multilayer film of the present disclosure has an effect of immediate release of the drug.

In another specific embodiment, in the drug-containing multilayer film of the present disclosure, the first polymer material in the drug-containing layer is a combination of polylactic acid and polyethylene glycol, and the polylactic acid is the second polylactic acid of which the viscosity may be about 1.1-3.0 dl/g, and the polyethylene glycol is a combination of the first polyethylene glycol of which the average molecular weight may be about 100-1,000 mentioned above and the second polyethylene glycol of which the average molecular weight may be about 4,000-10,000 mentioned above, and the weight ratio of the first polyethylene glycol to the second polyethylene glycol is about 1:0.01-20. The drug in the drug-containing layer may comprise the analgesic, antibiotic or a combination thereof mentioned above. Furthermore, in this specific embodiment, the drug-containing multilayer film has an effect of sustained release of the drug.

Furthermore, in one embodiment, the drug-containing layer of the drug-containing multilayer film may have at least one microstructures protruding from the other surface thereof, and the other surface of the drug-containing layer described herein is located on the opposite side of the anti-adhesion layer.

The microstructure mentioned may have an upper surface. The shape of the upper surface may be an arc-shaped surface, a flat surface, etc., but it is not limited thereto. The microstructures described herein do not include cone shapes or needle shapes.

In addition, the ratio of height to width of the bottom surface of the microstructure may be about 1:2-4, such as 1:1, 1:1.5, 1:2, but it is not limited thereto.

In one embodiment, the drug-containing layer mentioned above has a plurality of microstructures. In this embodiment, in the plurality of microstructures, a distance between the centers of two microstructures which are adjacent may be about 50-2,000 μm, such as about 75 μm, about 100 μm, about 150 μm, about 200 μm, about 220 μm, about 250 μm, about 300 μm, about 350 μm, about 400 μm, about 500 μm, about 1,000 μm, about 1,500 μm, but it is not limited thereto.

Moreover, in the foregoing embodiment in which the drug-containing layer has a plurality of microstructures, the density of the microstructures on the drug-containing layer may be about 0.1-100 microstructure/cm², such as about 1-30 microstructure/cm2, about 0.01 microstructure/cm², about 0.25 microstructure/cm², about 0.5 microstructure/cm², about 1 microstructure/cm², about 2 microstructure/cm², about 3 microstructure/cm², about 4 microstructure/cm², about 5 microstructure/cm², about 9 microstructure/cm², about 10 microstructure/cm², about 13 microstructure/cm², about 15 microstructure/cm², about 16 microstructure/cm², about 20 microstructure/cm², about 25 microstructure/cm², about 29 microstructure/cm², about 30 microstructure/cm², about 35 microstructure/cm², about 36 microstructure/cm², about 49 microstructure/cm², about 50 microstructure/cm², about 60 microstructure/cm², about 64 microstructure/cm², about 65 microstructure/cm², about 70 microstructure/cm², about 75 microstructure/cm², about 80 microstructure/cm², about 81 microstructure/cm², about 90 microstructure/cm², about 100 microstructure/cm², but it is also not limited thereto. The density of the microstructures is raised, and then the drug release efficiency is also be raised.

Furthermore, in the foregoing embodiment in which the drug-containing layer has a plurality of microstructures, the pattern arranged by the plurality of microstructures and/or the manner by which the plurality of microstructures of the drug-containing layer are arranged is not particularly limited, and can be adjusted as required. For example, a pattern arranged by the plurality of microstructures may comprise a rectangle, a circle, a triangle, an irregular shape, but it is not limited thereto, or the plurality of microstructures can be evenly distributed or unevenly distributed on the drug-containing layer. In one specific embodiment, the plurality of microstructures of the drug-containing layer mentioned above is arranged in a matrix.

Thickness of the drug-containing layer of the drug-containing multilayer film of the present disclosure may be about 5-55 μm, such as about 20-25 μm, but it is not limited thereto.

Moreover, the anti-adhesion layer of the drug-containing multilayer film of the present disclosure mentioned above may be composed of a second composition. The second composition mentioned above may comprise, but is not limited to, a second polymer material.

Examples of the second polymer material mentioned above may comprise, but is not limited to, polylactic acid, polyethylene glycol, or any combination thereof.

The viscosity of the polylactic acid in the examples of the second polymer material mentioned above may be about 1.1-3.0 dl/g, such as about 1.6-2.4 dl/g, but it is not limited thereto.

In addition, the polyethylene glycol in the examples of the second polymer material mentioned above may comprise polyethylene glycol with different average molecular weight and any combination thereof. For instance, the polyethylene glycol in the examples of the second polymer material mentioned above may comprise a third polyethylene glycol, a fourth polyethylene glycol or a combination thereof, wherein the third polyethylene glycol and the fourth polyethylene glycol may have different average molecular weights. The average molecular weight of the third polyethylene glycol may be about 100-1,000, such as about 285-315, but it is not limited thereto, and the average molecular weight of the fourth polyethylene glycol may be about 4,000-10,000, such as about 5,000-7,000, but it is also not limited thereto.

In one embodiment, in the anti-adhesion layer mentioned above, the second polymer material may comprise polylactic acid, and the viscosity of the polylactic acid may be about 1.1-3.0 dl/g, but it is not limited thereto.

In another embodiment, in the anti-adhesion layer mentioned above, the second polymer material may comprise both the polylactic acid and the polyethylene glycol. In this embodiment, the viscosity of the polylactic acid may be about 1.1-3.0 dl/g, and the polyethylene glycol may be the foregoing third polyethylene glycol, the foregoing fourth polyethylene glycol, or a combination thereof. In this embodiment, in the anti-adhesion layer mentioned above, the weight ratio of the polylactic acid to the polyethylene glycol may be about 1:0.01-0.5, such as about 1:0.05, about 1:0.1, about 1:0.11, about 1:0.2, about 1:0.24, about 1:0.25, about 1:0.3, about 1:0.4, about 1:0.45, but it is not limited thereto.

Furthermore, in one specific embodiment, the second polymer material may comprise both the polylactic acid and the polyethylene glycol, and the viscosity of the polylactic acid may be about 1.1-3.0 dl/g, and the polyethylene glycol may be the foregoing third polyethylene glycol or the foregoing fourth polyethylene glycol.

In another specific embodiment, the second polymer material may comprise both the polylactic acid and the polyethylene glycol, and the viscosity of the polylactic acid may be about 1.1-3.0 dl/g, and the polyethylene glycol may be a combination of the foregoing third polyethylene glycol and the foregoing fourth polyethylene glycol. In this specific embodiment, the weight ratio of the foregoing third polyethylene glycol to the foregoing fourth polyethylene glycol may be about 1:0.2-5, such as 1:0.25, 1:0.4, 1:0.5, 1:1, 1:2, 1:3, 1:4, but it is not limited thereto.

Thickness of the anti-adhesion layer of the drug-containing multilayer film of the present disclosure may be about 10-60 μm, such as about 25-30 μm, but it is not limited thereto.

Moreover, the drug-containing multilayer film of the present disclosure, in one embodiment, in addition to the drug-containing layer and the anti-adhesion layer mentioned above, may further comprise a protective layer over the other surface of the drug-containing layer, wherein the drug-containing layer is between the anti-adhesion layer and the protective layer.

The protective layer may be composed of a third composition. Third composition mentioned above may comprise a third polymer material, but it is not limited thereto.

Examples of the third polymer material may comprise, but is not limited to, polylactic acid, polyethylene glycol, or any combination thereof.

Examples of the polylactic acid in the third polymer material mentioned above may comprise polylactic acid with different viscosities and any combination thereof. For instance, the polylactic acid in the examples of the third polymer material mentioned above may comprise a third polylactic acid, a fourth polylactic acid or a combination thereof, wherein the third polylactic acid and the fourth polylactic acid may have different viscosities. The viscosity of the third polylactic acid may be about 0.05-1 dl/g, such as about 0.4-0.6 dl/g, but it is not limited thereto, and the viscosity of the fourth polylactic acid may be about 1.1-3.0 dl/g, such as about 1.6-2.4 dl/g, but it is also not limited thereto.

Moreover, the polyethylene glycol in the examples of the third polymer material may comprise polyethylene glycol with different average molecular weight and any combination thereof. For instance, the polyethylene glycol in the examples of the third polymer material may comprise a fifth polyethylene glycol, a sixth polyethylene glycol or a combination thereof, wherein the fifth polyethylene glycol and the sixth polyethylene glycol may have different average molecular weights. The average molecular weight of the fifth polyethylene glycol may be about 100-1,000, such as about 285-315, but it is not limited thereto, and the average molecular weight of the sixth polyethylene glycol may be about 4,000-10,000, such as about 5,000-7,000, but it is also not limited thereto.

In one embodiment, in the protective layer mentioned above, the third polymer material may comprise the polylactic acid. In this embodiment, the polylactic acid may be the foregoing the third polylactic acid, the foregoing fourth polylactic acid or a combination thereof.

In another embodiment, in the protective layer mentioned above, the third polymer material may comprise both the polylactic acid and the polyethylene glycol. In this embodiment, the polylactic acid may be the foregoing third polylactic acid or the foregoing fourth polylactic acid, and the polyethylene glycol may be the foregoing fifth polyethylene glycol, the foregoing sixth polyethylene glycol or a combination thereof. In this embodiment, in the protective layer mentioned above, the weight ratio of the polylactic acid to the polyethylene glycol may be about 1:0.05-1, such as about 1:0.1, about 1:0.2, about 1:0.25, about 1:0.3, about 1:0.4, about 1:0.43, about 1:0.5, about 1:0.75, but it is not limited thereto.

Furthermore, in one specific embodiment, the third polymer material may comprise both the polylactic acid and the polyethylene glycol, and the polylactic acid may be the foregoing third polylactic acid or the foregoing fourth polylactic acid, and the polyethylene glycol may be the foregoing fifth polyethylene glycol or the foregoing sixth polyethylene glycol.

In another specific embodiment, the third polymer material may comprise both the polylactic acid and the polyethylene glycol, and the polylactic acid may be the foregoing third polylactic acid or the foregoing fourth polylactic acid, and the polyethylene glycol may be the combination of the foregoing fifth polyethylene glycol and the foregoing sixth polyethylene glycol. In this specific embodiment, the weight ratio of the foregoing fifth polyethylene glycol to the foregoing sixth polyethylene glycol may be about 1:0.05-20, such as 1:0.1, 1:0.2, 1:0.5, 1:1, 1:5, 1:10, 1:15, but it is not limited thereto.

Thickness of the protective layer of the drug-containing multilayer film of the present disclosure may be about 1-20 μm, such as about 1-13 μm, but it is not limited thereto.

In one embodiment, the drug-containing multilayer film of the present disclosure has the drug-containing layer and the anti-adhesion layer, and the thickness thereof may be about 15-115 μm, such as about 45-60 μm, but it is not limited thereto. In another embodiment, the drug-containing multilayer film of the present disclosure has the drug-containing layer, the anti-adhesion layer and the protective layer, and the thickness thereof may be may be about 16-135 μm, such as about 45-70 μm, but it is not limited thereto.

The present disclosure also provided a method for forming a drug-containing multilayer film which may be used for forming the drug-containing multilayer film of the present disclosure mentioned above.

The method for forming a drug-containing multilayer film of the present disclosure mentioned above may comprise, but is not limited to, the following manner (a), or manner (b).

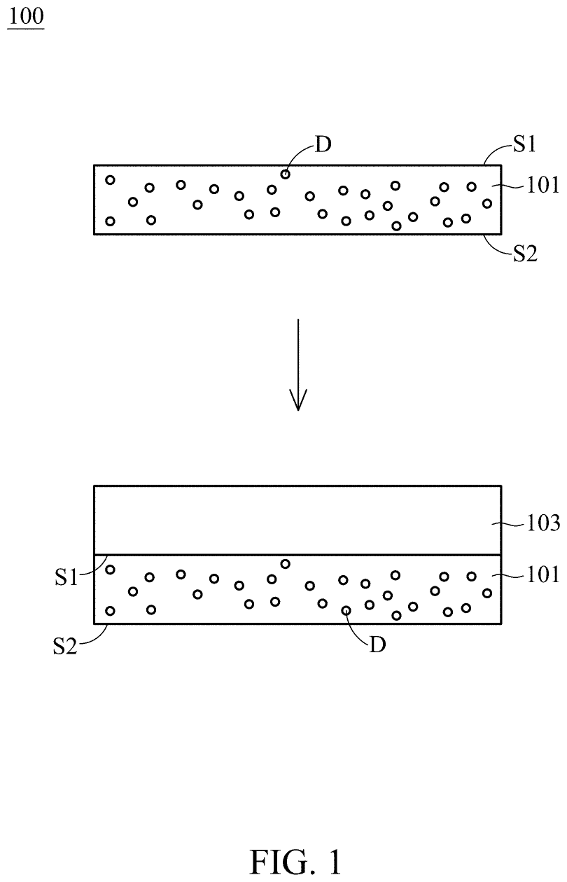

Refer to FIG. 1. FIG. 1 shows one embodiment of the method for forming a drug-containing multilayer film of the present disclosure, such as manner (a).

The manner (a) may comprise the following steps, but it is not limited thereto.

First, a first solution is dried to form a film to form a drug-containing layer 101. The drug-containing layer contains a drug D, and has a surface S1 and the other surface S2.

Next, a second solution is dried to form a film over the surface S1 of the drug-containing layer 101 to form an anti-adhesion layer 103, and the preparation of a drug-containing multilayer film 100 of one embodiment of the present disclosure is completed.

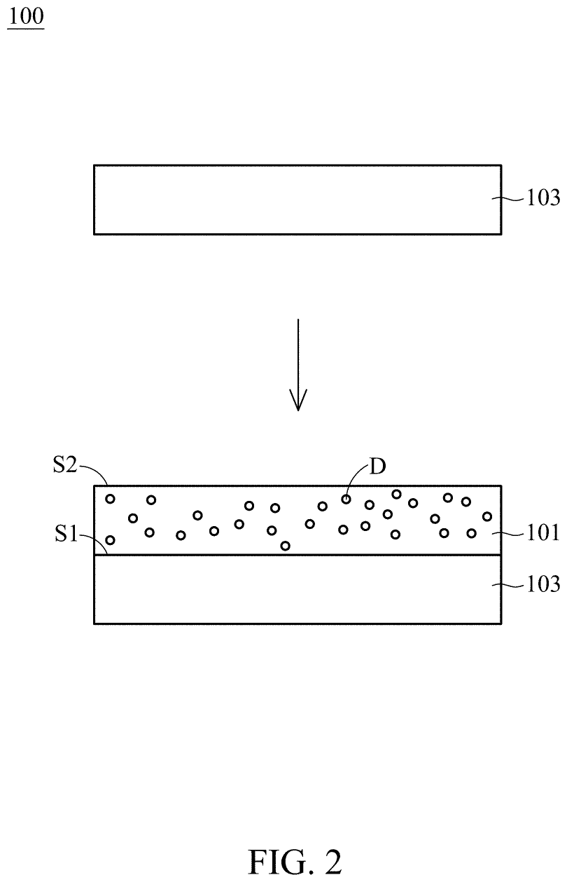

Furthermore, refer to FIG. 2. FIG. 2 shows another embodiment of the method for forming a drug-containing multilayer film of the present disclosure, such as manner (b).

The manner (b) may comprise the following steps, but it is not limited thereto.

First, a second solution is dried to form a film to form an anti-adhesion layer 103.

Next, a first solution is dried to form a film over anti-adhesion layer 103 to form a drug-containing layer 101, and the preparation of a drug-containing multilayer film 100 of one embodiment of the present disclosure is completed. The drug-containing layer contains a drug D.

The solute of the first solution mentioned above may comprise a first composition. The first composition mentioned above may comprise, but is not limited to, a first polymer material and a drug. Moreover, examples of the solvent of the foregoing solution may comprise, but are not limited to, dichloromethane, tetrahydrofuran, chloroform, but they are not limited thereto.

The content of the first polymer material in the first solution mentioned above may be 5-30 wt %, such as about 10-25 wt %, about 15 wt %, about 20 wt %, but it is not limited thereto. Moreover, the weight ratio of the first polymer material mentioned above to the drug mentioned above may be about 1:0.01-0.3, such as about 1:0.015, 1:0.02, 1:0.05, 1:0.1, 1:0.15, 1:0.2, 1:0.25, but it is also not limited thereto.

The solute of the second solution mentioned above may comprise a second composition. The second composition mentioned above may comprise, but is not limited to, a second polymer material, but it is not limited thereto. Examples of the solvent of the foregoing second solution may comprise, but are not limited to, dichloromethane, tetrahydrofuran, chloroform.

A content of the second polymer material mentioned above in the second solution mentioned above may be about 5-30 wt %, such as about 10-25 wt %, about 15 wt %, about 20 wt %, but it is not limited thereto.

The first composition, the first polymer material and the drug involved in the first solution, may be the same as the first composition, the first polymer material and the drug of the drug-containing layer of the drug-containing multilayer film of the present disclosure mentioned above, respectively, and thus all relevant interpretations thereof can be referred to the related descriptions for the first composition, the first polymer material and the drug of the drug-containing layer of the drug-containing multilayer film of the present disclosure in the preceding paragraphs and are not repeated herein.

Similarly, the second composition and the second polymer material involved in the second solution, may be the same as the second composition and the second polymer material of the anti-adhesion layer of the drug-containing multilayer film of the present disclosure mentioned above, respectively, and thus all relevant interpretations thereof can be referred to the related descriptions for the second composition and the second polymer material of the anti-adhesion layer of the drug-containing multilayer film of the present disclosure in the preceding paragraphs and are not repeated herein.

Moreover, in one embodiment, in the foregoing manner (a), a film forming method for the drug-containing layer may comprise, but is not limited to the following steps.

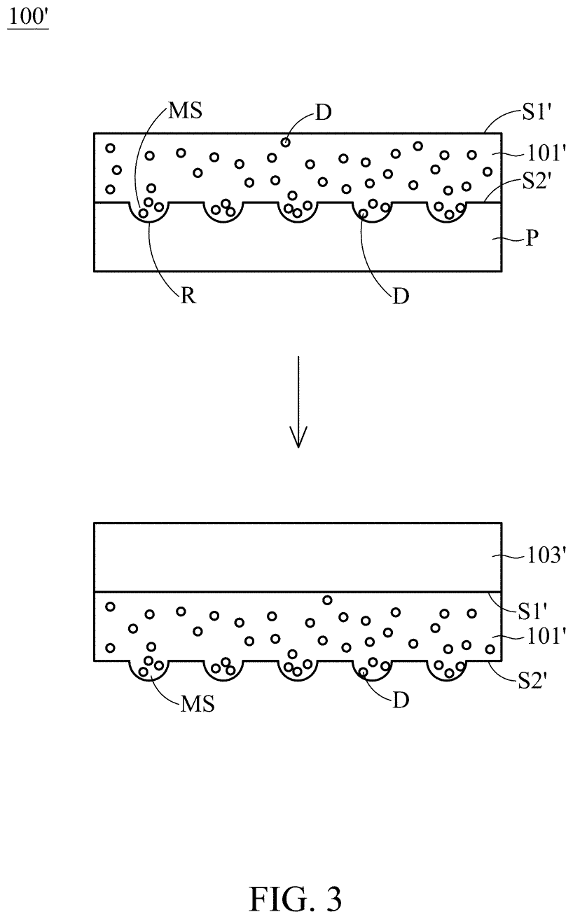

Refer to FIG. 3. The first solution is poured on a plate P with at least one recess R, and dried to form a film on the plate P with at least one recess R to form a drug-containing layer 101′ and make the drug-containing layer 101′ have at least one microstructure MS protruding from the surface S2′ thereof.

In this embodiment, the anti-adhesion layer 103′ is formed over the surface S1′ of the drug-containing layer 101′, subsequently.

The recess of the plate mentioned may have an upper surface. The shape of the upper surface may be an arc-shaped surface, a flat surface, etc., but it is not limited thereto. The shape of the microstructure of the drug-containing layer mentioned above is formed to correspond to the shape of the recess of the plate.

The aspect ratio of the recess mentioned above may be about 1:2-4, such as 1:1, 1:1.5, 1:2, but it is not limited thereto.

In one embodiment, the plate mentioned above has a plurality of recesses. In this embodiment, in the plurality of recesses, a distance between the centers of two recesses which are adjacent may be about 50-2,000 μm, such as about 75 μm, about 100 μm, about 150 μm, about 200 μm, about 220 μm, about 250 μm, about 300 μm, about 350 μm, about 400 μm, about 500 μm, about 1,000 μm, about 1,500 μm, but it is not limited thereto.

Moreover, in the foregoing embodiment in which the plate has a plurality of recesses, density of the recesses on the plate may be about 0.1-100 recess/cm², such as about 1-30 recess/cm², about 0.01 recess/cm², about 0.25 recess/cm², about 0.5 recess/cm², about 1 recess/cm², about 2 recess/cm², about 3 recess/cm², about 4 recess/cm², about 5 recess/cm², about 9 recess/cm², about 10 recess/cm², about 13 recess/cm², about 15 recess/cm², about 16 recess/cm², about 20 recess/cm², about 25 recess/cm², about 29 recess/cm², about 30 recess/cm², about 35 recess/cm², about 36 recess/cm², about 49 recess/cm², about 50 recess/cm², about 60 recess/cm², about 64 recess/cm², about 65 recess/cm², about 70 recess/cm², about 75 recess/cm², about 80 recess/cm², about 81 recess/cm², about 90 recess/cm², about 100 recess/cm², but it is also not limited thereto.

Furthermore, in the foregoing embodiment in which the plate has a plurality of recesses, the pattern arranged by the plurality of recesses and/or the manner by which the plurality of recesses of the plate are arranged is not particularly limited, and can be adjusted as required. For example, a pattern arranged by the plurality of recesses may comprise a rectangle, a circle, a triangle, an irregular shape, but it is not limited thereto, or the plurality of recesses can be evenly distributed or unevenly distributed on the plate. In one specific embodiment, the plurality of recesses of the plate mentioned above is arranged in a matrix.

In addition, material of the foregoing plate is not particularly limited, as long as it can form recesses with desired shape thereon without being dissolved or eroded by the first solution, and the formed film can be peeled off from it without breaking the film. Examples of the material of the foregoing plate may comprise stainless steel, glass, Teflon, but they are not limited thereto.

The microstructure of the drug-containing layer formed corresponding to the recess of the plate described herein may be the same as the microstructure of the drug-containing layer of the drug-containing multilayer film of the present disclosure mentioned above, and thus all relevant interpretation thereof can be referred to the related description for the microstructure of the drug-containing layer of the drug-containing multilayer film of the present disclosure in the preceding paragraphs and are not repeated herein.

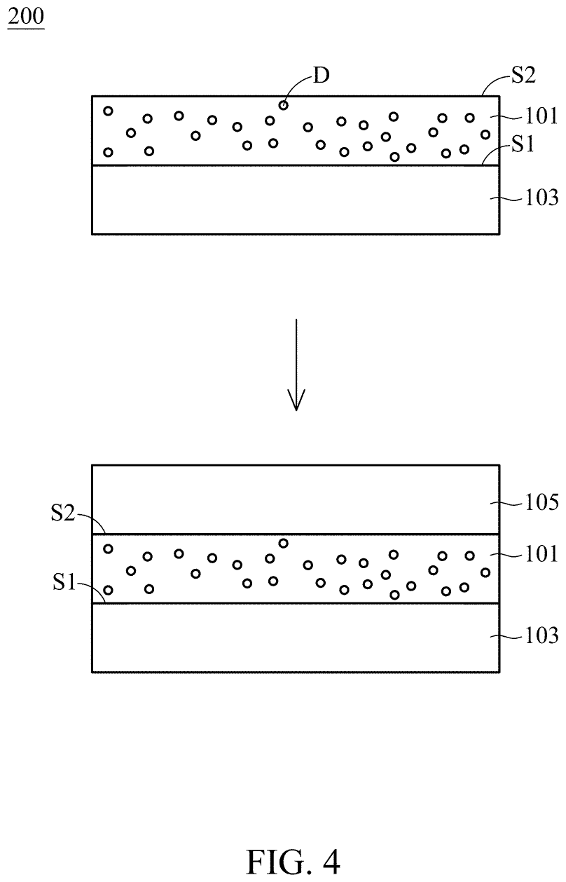

Furthermore, refer to FIG. 4. In one embodiment the method for forming a drug-containing multilayer film of the present disclosure, the manner (a) and the manner (b) may further comprise, but is not limited to, after forming both of the drug-containing layer 101 and the anti-adhesion layer 103, a third solution is dried to form a film on the drug-containing layer 101 to form a protective layer 105 to form a drug-containing multilayer film 200, wherein the drug-containing layer is between the anti-adhesion layer and the protective layer.

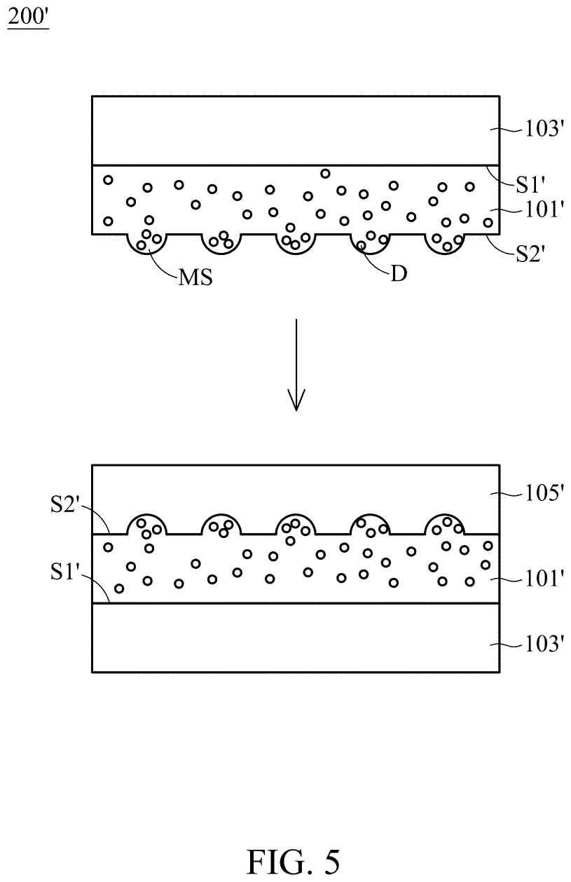

In addition, refer to FIG. 5. In a specific embodiment of the method for forming a drug-containing multilayer film disclosed herein, under a condition that formed drug-containing layer has microstructures, after both of the drug-containing layer 101′ and the anti-adhesion layer 103′ are formed, the third solution is dried to form a film over the surface S2′ having the microstructures of the drug-containing layer 101′ to form a protective layer 105′ to form a drug-containing multilayer film 200′, wherein the drug-containing layer 101′ is between the anti-adhesion layer 103′ and the protective layer 105′.

The solute of the third solution mentioned above may comprise a third composition. The third composition mentioned above may comprise, but is not limited to, a third polymer material, but it is not limited thereto. Examples of the solvent of the foregoing third solution may comprise, but are not limited to, dichloromethane, tetrahydrofuran, chloroform.

Moreover, the content of the third polymer material mentioned above in the third solution mentioned above may be about 5-30 wt %, such as about 10-25 wt %, about 15 wt %, about 20 wt %, but it is not limited thereto.

The third composition and the third polymer material involved in the third solution, may be the same as the third composition and the third polymer material of the protective layer of the drug-containing multilayer film of the present disclosure mentioned above, respectively, and thus all relevant interpretations thereof can be referred to the related descriptions for the third composition and the third polymer material of the protective layer of the drug-containing multilayer film of the present disclosure in the preceding paragraphs and are not repeated herein.

### EXAMPLES

A. Anti-Adhesion Film

A-1. Preparation of Anti-Adhesion Film

Each anti-adhesion film was prepared according to the formula in the following Table 1.

TABLE 1

| | Content in solution (wt %) | | |
|---|---|---|---|
| | Polylactic acid (poly(DL- | | |
| Name of anti- | lactide)) | Polyethylene | Polyethylene |
| adhesion film | (Viscosity: 1.6-2.4 dl/g) | glycol 300 | glycol 6000 |
| AM | 8.8 | 1.1 | 1.1 |
| BM | 9.9 | 1.1 | — |

Polyethylene glycol 300: the average molecular weight of polyethylene glycol is 285-315;
Polyethylene glycol 6000: the average molecular weight of polyethylene glycol is 5,000-10,000;
—: No addition.

First, polylactic acid and polyethylene glycol were dissolved in dichloromethane and mixed well to form a solution with a total polymer content of 11 wt %.

Next, this solution was poured on a glass plate, and evenly coated on the surface of the glass plate with an automatic film scraping machine and a coating scraper (scale 230 μm).

After that, the glass plate mentioned above was placed in a fume hood to volatilize the dichloromethane to form an anti-adhesion film.

A-2. Anti-Adhesion Test for Anti-Adhesion Film

A-2-1. Cell Assay

1. Methods

Cells used in this assay were L929 cells.

(1) Observation of Cell Appearance

First, the anti-adhesive film (BM) was cut to an appropriate size and placed in wells of a 24-well plate, and cell suspension was added to the wells. Subsequently, the 24-well plate was moved to a 5% CO₂ and 37° C. cell incubator for culturing. After culturing for 24 hours, cell appearance was photographed by a microscope. In the control group, only the cell suspension was added to the culture plate.

(2) Cell Viability Analysis

Cell viability analysis was performed by MTT assay.

First, the anti-adhesive film (BM) was cut to an appropriate size and placed in wells of a 96-well plate, and cell suspension was added to the wells. Subsequently, the 96-well plate was moved to a 5% CO₂ and 37° C. cell incubator for culturing. In the control group, only the cell suspension was added to the culture plate. The blank group was simply culture medium.

After culturing for 24 hours, the original medium in the culture plate was removed, and then fresh medium containing MTT reagent was added to the culture plate. After that, the culture plate was placed to a 5% CO₂ and 37° C. cell incubator for culturing four hours.

Finally, the medium in the culture plate was removed and a mixture solution of HCl/iso-propanol was added to the culture plate to dissolve the products in the cells, and absorbance of each well at 570 nm was determined by a microplate reader.

2. Results

(1) Observation of Cell Appearance

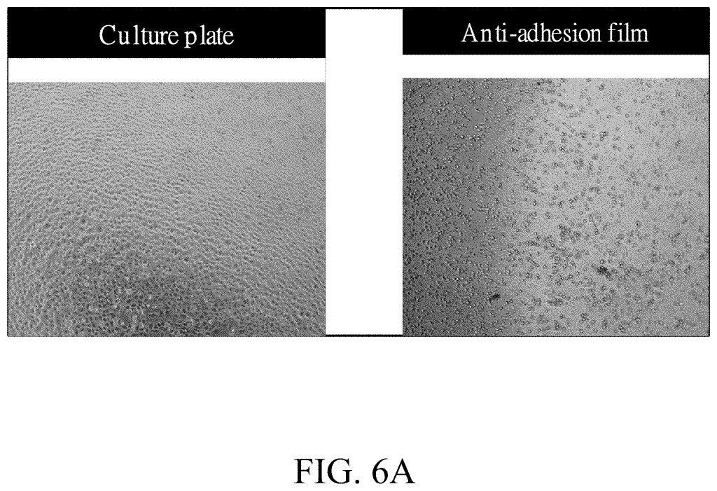

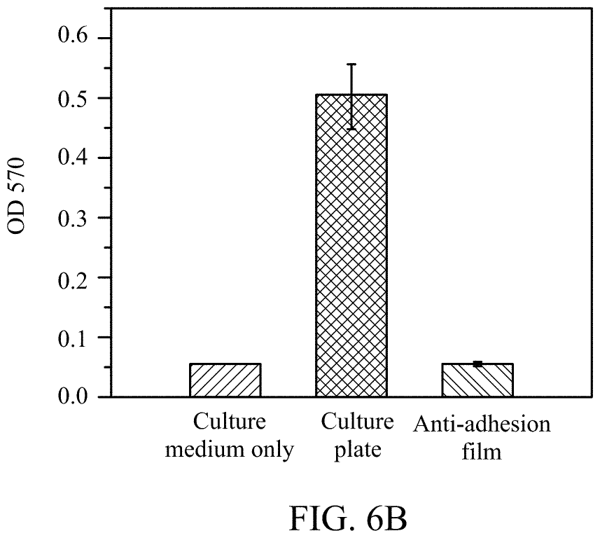

The result is shown in FIG. 6A.

FIG. 6A shows that, in the control group which only uses culture medium for culturing, the cells attach to the culture plate and grow, but in the experimental group in which the anti-adhesion film is present, cell almost cannot attach to the anti-adhesion film and only can suspend in the culture medium. This result shows that the anti-adhesion film in the present disclosure indeed has an effect of preventing cell attachment and growth.

(2) Cell Viability Analysis

The result is shown in FIG. 6B.

FIG. 6B shows that the absorbance at 570 nm of the control group which only uses culture medium for culturing can reach to about 0.5. In contract, the absorbance at 570 nm of the experimental group in which the anti-adhesion film is present is approximately equal to the absorbance at 570 nm of culture medium only, and this represents that the cells in the experimental group almost cannot survive. This assay also proved that the anti-adhesion film in the present disclosure can prevent cell attachment and growth.

A-2-2. Animal Experiment

1. Methods

Anti-adhesion test was performed by male Sprague-Dawley rats.

First, a rat was anesthetized by intraperitoneal injection of Zotetil (25 mg/0.5 ml/kg) and Xylazine (10 mg/0.5 ml/kg).

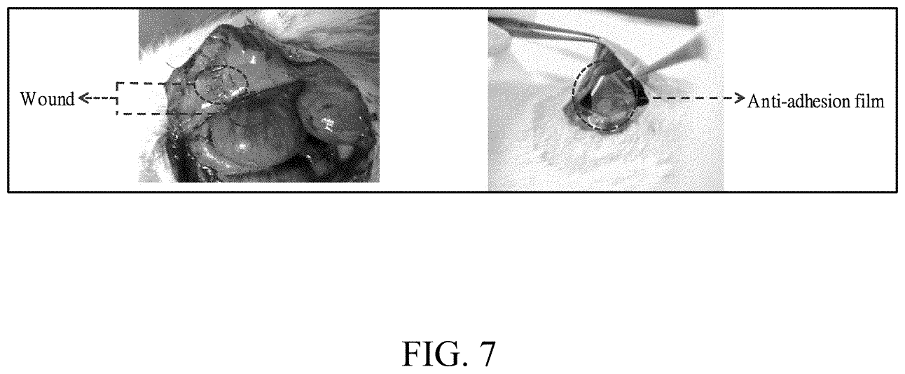

Next, surgery was performed on the right abdomen of the rat to cut an opening with a size of 1.5 cm² and to create a scratch with a small area on the cecum and the abdominal wall adjacent to the cecum (referring to the left side photograph of FIG. 7).

After that, the rats were divided to 3 groups which were the control group, the experimental group 1 and the experimental group 2, respectively, and each group used 10 rats. In the control group, the rats were not implanted with any films. In the experimental group 1, the rats were implanted with the anti-adhesion film AM prepared above, and in the experimental group 2, the rats were implanted with the anti-adhesion film BM prepared above. In experimental group 1 and the experimental group 2, the films were implanted between the cecum and the abdominal wall of the rats (referring to the right side photograph of FIG. 7). After the surgery was completed, the cecum and the abdominal wall of the rat were sutured.

After the rats subjected to the surgery were kept for feeding for one month, the rats were anesthetized to photograph of the cecum and the abdominal wall of the surgical site and observe the degree of adhesion. After that, based on the criteria shown in the following Table 2, the severity of adhesion between the cecum and the abdominal wall of each group of rats was determined and analyzed.

TABLE 2
Severity of adhesion based on grade of adhesion and
percentage of area of injured cecum surface (Shmuel
Avital. et al., Dis Colon Rectum, 48: 153-157, 2005)

| Severity | Grade | % area with adhesion |
|---|---|---|
| Absent | 0 | 0 |
| Mild | 1 | 1-100 |
| Moderate | 2 | 1-100 |
| | 3 | <100 |
| Severe | 3 | 100 |

0 = No adhesion; 1 = Thin, filamentous, easily detachable adhesion; 2 = Thick adhesion, difficult to be cut apart, will not tear organs when separated; 3 = Thick adhesion, cannot be cut apart, will tear organs when separated.
“Absence of adhesion” is defined as the absence of adhesion from the cecum to the abdominal wall.
“Mild adhesion” is defined as all Grade 1 adhesion between the cecum and the abdominal wall.
“Moderate adhesion” includes all Grade 2 adhesion between the cecum and the abdominal wall and any Grade 3 adhesions involving abraded wall of the cecum having less than 100% adhesion area.
“Severe adhesion” belongs to any Grade 3 adhesions involving abraded wall of the cecum having 100% adhesion area.

2. Results

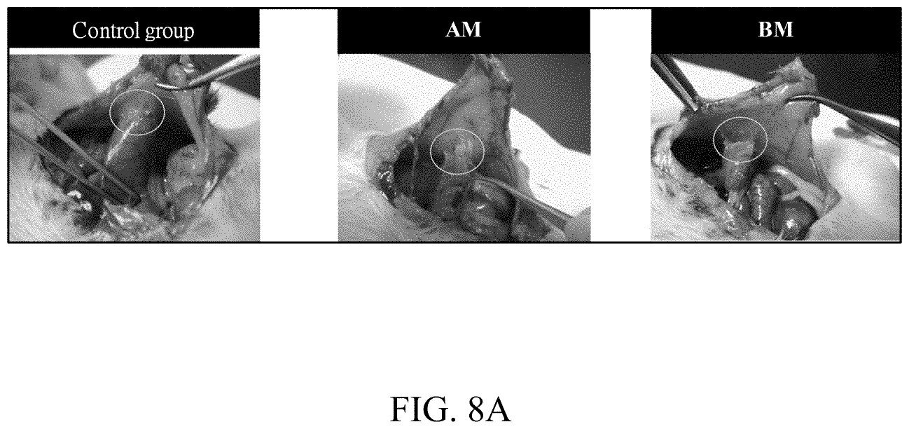

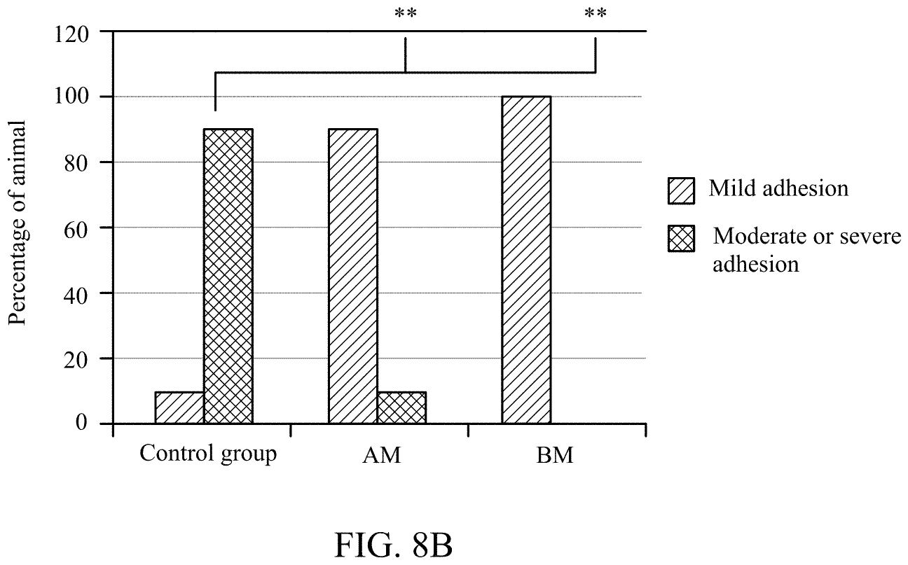

The results are shown in FIG. 8A and FIG. 8B.

FIG. 8A shows that there was severe adhesion between the cecum and the abdominal wall of rats in the control group, and only a small amount of filamentous adhesion between the cecum and the abdominal wall of rats in the experimental group 1 (anti-adhesion film AM), and only slight adhesion between the cecum and the abdominal wall of rats in the experimental group 2 (anti-adhesion film BM). Specifically, compared to the control group in which there was many connective tissues between the cecum and the abdominal wall, the rats in experimental group 1 and experimental group 2 had the anti-adhesion film of the present disclosure as a barrier, and thus the connective tissue was difficult to form between the cecum and the abdominal wall, so the degree of adhesion is not obvious.

FIG. 8B shows the percentage of rats with mild adhesion and moderate or severe adhesion in each group. FIG. 8B shows that in the control group, 80% of the rats had moderate or severe adhesion, in the experimental group 1 (AM), only 15% of the rats are classified as moderate adhesion belonging to Grades 2 to 3, and the rest are all classified as mild adhesion belonging to Grade 1, and in experimental group 2 (BM), even all rats are classified as mild adhesion belonging to Grade 1.

Based on the results mentioned above, it is known that the films formed by polylactic acid of high viscosity (viscosity 1.6-2.4 dl/g) can indeed achieve anti-adhesion effect.

B. Drug-Containing Multilayer Film

B-1. Preparation of Drug-Containing Multilayer Film

B-1-1. Preparation of Drug-Containing Multilayer Film (Having Drug-Containing Layer with Flat Surface)

(a) Preparation Manner 1

Each drug-containing multilayer film was prepared according to the formula in the following Table 3.

TABLE 3

| | | Content in solution (wt %) | | | | |
|---|---|---|---|---|---|---|
| | | Polylactic | Polylactic | | | |
| | | acid (poly(DL- | acid (poly(DL- | | | |
| | | lactide)) | lactide)) | | | |
| | | (Viscosity: | (Viscosity: | Polyethylene | Polyethylene | Lidocaine |
| Name | | 0.4-0.6 dl/g) | 1.6-2.4 dl/g) | glycol 300 | glycol 6000 | hydrochloride |
| M-01 | Anti-adhesion | — | 11 | — | — | — |
| | layer | | | | | |
| | Drug-containing | 20 | — | — | — | 4 |
| | layer | | | | | |
| M-02 | Anti-adhesion | — | 11 | — | — | — |
| | layer | | | | | |
| | Drug-containing | 14 | — | 6 | — | 4 |
| | layer | | | | | |
| M-03 | Anti-adhesion | — | 11 | — | — | — |
| | layer | | | | | |
| | Drug-containing | 14 | — | — | 6 | 4 |
| | layer | | | | | |
| M-04 | Anti-adhesion | — | 11 | — | — | — |
| | layer | | | | | |
| | Drug-containing | 14 | — | 5 | 1 | 4 |
| | layer | | | | | |
| M-05 | Anti-adhesion | — | 11 | — | — | — |
| | layer | | | | | |
| | Drug-containing | 14 | — | 3 | 3 | 4 |
| | layer | | | | | |
| M-06 | Anti-adhesion | — | 11 | — | — | — |
| | layer | | | | | |
| | Drug-containing | 14 | — | 1 | 5 | 4 |
| | layer | | | | | |
| M-07 | Anti-adhesion | — | 11 | — | — | — |
| | layer | | | | | |
| | Drug-containing | 14 | — | 6 | — | 4 |
| | layer | | | | | |
| | Protective layer | 20 | — | — | — | — |
| M-08 | Anti-adhesion | — | 11 | — | — | — |
| | layer | | | | | |
| | Drug-containing | 14 | — | 6 | — | 4 |
| | layer | | | | | |
| | Protective layer | 14 | — | 6 | — | — |
| M-09 | Anti-adhesion | — | 11 | — | — | — |
| | layer | | | | | |
| | Drug-containing | 14 | — | 6 | — | 4 |
| | layer | | | | | |
| | Protective layer | 14 | — | — | 6 | — |
| M-10 | Anti-adhesion | — | 11 | — | — | — |
| | layer | | | | | |
| | Drug-containing | 14 | — | 6 | — | 4- |
| | layer | | | | | |
| | Protective layer | 14 | — | 3 | 3 | — |
| M-11 | Anti-adhesion | — | 11 | — | — | — |
| | layer | | | | | |
| | Drug-containing | — | 14 | 3 | 3 | 4 |
| | layer | | | | | |
| M-12 | Anti-adhesion | — | 11 | — | — | — |
| | layer | | | | | |
| | Drug-containing | — | 14 | 3 | 3 | 4 |
| | layer | | | | | |
| | Protective layer | — | 14 | 3 | 3 | — |

(1) Preparation of Anti-Adhesion Layer

First, polylactic acid and polyethylene glycol were dissolved in dichloromethane and mixed well to form a solution with a total polymer content of 11 wt %.

Next, this solution was poured on a glass plate, and evenly coated on the surface of the glass plate with an automatic film scraping machine and a coating scraper (scale 230 μm).

After that, the glass plate mentioned above was placed in a fume hood to volatilize the dichloromethane to form an anti-adhesion layer.

(2) Preparation of Drug-Containing Layer

Polylactic acid and dichloromethane were mixed well to form a polylactic acid solution.

If the ingredients did not include polyethylene glycol, then lidocaine hydrochloride was added to the polylactic acid solution mentioned above and mixed uniformly to form a mixture solution. If the ingredients included polyethylene glycol, first, polyethylene glycol was added to the polylactic acid solution mentioned above and mixed well, and then lidocaine hydrochloride was added to this polylactic acid and polyethylene glycol solution and mixed well to form a mixture solution. In the mixture solution, the total polymer content was 20 wt %, and the drug content is 20% of the polymer content.

Next, this solution was poured on the surface of the foregoing anti-adhesion layer formed on the glass plate, and evenly coated on the anti-adhesion layer with an automatic film scraping machine and a coating scraper (scale 200 μm).

After that, the glass plate mentioned above was placed in a fume hood to volatilize the dichloromethane to form a drug-containing layer on the anti-adhesion film. If the drug-containing multilayer film did not have a protective layer, then the preparation of the drug-containing multilayer film was completed at this step. If the drug-containing multilayer film had a protective layer, the following procedure was continued.

(3) Protective Layer

Polylactic acid and polyethylene glycol were dissolved in dichloromethane and mixed well to form a polylactic acid solution.

If the ingredients did not include polyethylene glycol, then the polylactic acid solution was poured on the drug-containing layer in the two layers of anti-adhesion layer and drug-containing layer on the glass plate, and evenly coated on the surface of the drug-containing layer with an automatic film scraping machine and a coating scraper (scale 100 μm).

If the ingredients included polyethylene glycol, polyethylene glycol was added to the polylactic acid solution to the polylactic acid solution mentioned above and mixed well to form a mixture solution with a total polymer content of 20 wt %. Next, the mixture solution was poured on the drug-containing layer in the two layers of anti-adhesion layer and drug-containing layer on the glass plate, and evenly coated on the surface of the drug-containing layer with an automatic film scraping machine and a coating scraper (scale 100 μm).

After that, the glass plate mentioned above was placed in a fume hood to volatilize the dichloromethane to form a protective layer on the drug-containing layer to complete the preparation of drug-containing multilayer film.

(b) Preparation Manner 2

Drug-containing multilayer film RM was prepared according to the formula in the following Table 4.

TABLE 4

| | | Content in solution (wt %) | | | | |
|---|---|---|---|---|---|---|
| | | Polylactic | Polylactic | | | |
| | | acid (poly(DL- | acid (poly(DL- | | | |
| | | lactide)) | lactide)) | | | |
| | | (Viscosity: | (Viscosity: | Polyethylene | Polyethylene | Lidocaine |
| Name | | 0.4-0.6 dl/g) | 1.6-2.4 dl/g) | gly 300 | gly 6000 | hydrochloride |
| RM | Drug-containing | — | 14 | 3 | 3 | 4 |
| | layer | | | | | |
| | Anti-adhesion | — | 11 | — | — | — |
| | layer | | | | | |

(1) Preparation of Drug-Containing Layer

First, polylactic acid and dichloromethane were mixed well to form a polylactic acid solution.

Polyethylene glycol was added to the polylactic acid solution mentioned above and mixed well, and then lidocaine hydrochloride was added to this polylactic acid and polyethylene glycol solution and mixed well to form a mixture solution. In the mixture solution, the total polymer content was 20 wt %, and the drug content is 20% of the polymer content.

Next, this solution was poured on a glass plate, and evenly coated on the surface of the glass plate with an automatic film scraping machine and a coating scraper (scale 200 μm).

After that, the glass plate mentioned above was placed in a fume hood to volatilize the dichloromethane to form a drug-containing layer.

(2) Preparation of Anti-Adhesion Layer

Polylactic acid and polyethylene glycol were dissolved in dichloromethane and mixed well to form a solution with a total polymer content of 11 wt %.

Next, this solution was poured on the foregoing drug-containing layer formed on the glass plate, and evenly coated on the surface of the drug-containing with an automatic film scraping machine and a coating scraper (scale 230 μm).

After that, the glass plate mentioned above was placed in a fume hood to volatilize the dichloromethane to form an anti-adhesion layer to complete the preparation of drug-containing multilayer film RM.

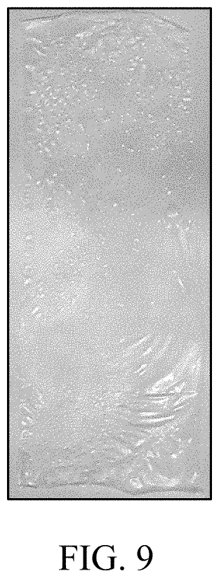

Drug-containing multilayer film RM can be indeed formed by the preparation manner 2, and can be completely peeled from the glass plate, referring to FIG. 9.

B-1-2. Preparation of Drug-Containing Multilayer Film (Having Drug-Containing Layer with Microstructures)

Each drug-containing multilayer film was prepared according to the formula in the following Table 5.

TABLE 5

| | | Distance between | | Content in solution (wt %) | | | | |
|---|---|---|---|---|---|---|---|---|
| | Density of | the centers of | | Polylactic acid | Polylactic acid | | | |
| | microstructure | two microstructures | | (poly(DL-lactide)) | (poly(DL-lactide)) | | | |
| | (microstructure/ | which are adjacent | | (Viscosity: | (Viscosity: | Polyethylene | Polyethylene | Lidocaine |
| Name | cm²) | (μm) | | 0.4-0.6 dl/g) | 1.6-2.4 dl/g) | glycol 300 | glycol 6000 | hydrochloride |
| MS-1 | 29 | 200 | Drug- | — | 14 | 3 | 3 | 4 |
| | | | containing | | | | | |
| | | | layer | | | | | |
| | | | Anti- | — | 11 | — | — | — |
| | | | adhesion | | | | | |
| | | | layer | | | | | |
| MS-2 | 13 | 350 | Drug- | — | 14 | 3 | 3 | 4 |
| | | | containing | | | | | |
| | | | layer | | | | | |
| | | | Anti- | — | 11 | — | — | — |
| | | | adhesion | | | | | |
| | | | layer | | | | | |
| MS-3 | 3 | 1000 | Drug- | — | 14 | 3 | 3 | 4 |
| | | | containing | | | | | |
| | | | layer | | | | | |
| | | | Anti- | — | 11 | — | — | — |
| | | | adhesion | | | | | |
| | | | layer | | | | | |
| MS- | 29 | 200 | Drug- | — | 14 | 6 | — | 4 |
| 1-B | | | containing | | | | | |
| | | | layer | | | | | |
| | | | Anti- | — | 11 | — | — | — |
| | | | adhesion | | | | | |
| | | | layer | | | | | |
| | | | Protective | — | 14 | 3 | 3 | — |
| | | | layer | | | | | |
| MS- | 13 | 350 | Drug- | — | 14 | 6 | — | 4 |
| 2-B | | | containing | | | | | |
| | | | layer | | | | | |
| | | | Anti- | — | 11 | — | — | — |
| | | | adhesion | | | | | |
| | | | layer | | | | | |
| | | | Protective | — | 14 | 3 | 3 | — |
| | | | layer | | | | | |

(1) Preparation of Drug-Containing Layer

(i) Making of Stainless Steel Mold

A surface of a stainless steel plate is processed so that at least one recess is generated on the surface thereof. The number of recess, the shape of the recess and the pattern arranged by the plurality of recesses can be set according to practical need. Therefore, the specifications of the mold can be customized, and a film containing a specific microstructure can be produced by mold turning.

In this preparation, three stainless steel plate molds were made, and the recesses on the surface were all recesses having an upper surface with a circular arc surface, and the densities of recesses were 29 recess/cm², 13 recess/cm² and 3 recess/cm², respectively.

(ii) Preparation of Drug-Containing Layer

polylactic acid and dichloromethane were mixed well to form a polylactic acid solution.

Polyethylene glycol was added to the polylactic acid solution mentioned above and mixed well, and then lidocaine hydrochloride was added to this polylactic acid and polyethylene glycol solution and mixed well to form a mixture solution. In the mixture solution, the total polymer content was 20 wt %, and the drug content is 20% of the polymer content.

Next, this solution was poured on the stainless steel plate mold, and evenly coated on the stainless steel plate mold with an automatic film scraping machine and a coating scraper (scale 200 μm).

After that, the stainless steel plate mold mentioned above was placed in a fume hood to volatilize the dichloromethane (about 24 hours) to form a drug-containing layer with microstructures.

(2) Preparation of Anti-Adhesion Layer

Polylactic acid and polyethylene glycol were dissolved in dichloromethane and mixed well to form a solution with a total polymer content of 11 wt %.

Next, this solution was poured on the foregoing drug-containing layer formed on the stainless steel plate mold, and evenly coated on the surface of the drug-containing layer with an automatic film scraping machine and a coating scraper (scale 230 μm).

After that, the stainless steel plate mold mentioned above was placed in a fume hood to volatilize the dichloromethane to form a drug-containing layer on the anti-adhesion film. If the drug-containing multilayer film did not have a protective layer, then the preparation of the drug-containing multilayer film was completed at this step. If the drug-containing multilayer film had a protective layer, with the following procedure was continued.

(3) Protective Layer

Polylactic acid and polyethylene glycol were dissolved in dichloromethane and mixed well to form a polylactic acid solution. In the mixture solution, the total polymer content was 20 wt %.

Next, the two layers of the drug-containing layer and the anti-adhesion layer on the stainless steel plate are removed from the stainless steel plate mold, and the two layers of the drug-containing layer and the anti-adhesion layer were placed on the surface of a glass plate with the microstructure of the drug-containing layer facing up.

Next, the mixture solution was poured on the surface having microstructures in the two layers of anti-adhesion layer and drug-containing layer, and evenly coated on the surface of the drug-containing layer with an automatic film scraping machine and a coating scraper (scale 100 μm).

After that, the glass plate mentioned above was placed in a fume hood to volatilize the dichloromethane to form a protective layer on the drug-containing layer to complete the preparation of drug-containing multilayer film.

B-2. In Vitro Drug Release Test of Drug-Containing Multilayer Films

1. Methods

The drug-containing multilayer film was cut into an appropriate size and placed in a tissue embedding cassette to fix the periphery of the film to avoid film curling.

Next, the tissue embedding cassette was placed in a glass bottle containing 40 ml phosphate buffered saline (PBS), and the glass bottle was shaken at a rate of 100 rpm to enable the film to simulate the dynamic environment in the body to release the drug, and sampling was performed at specific time points.

The release tests were divided into an immediate release test and a sustained release test. The sampling times of the immediate release test were 0.5, 1, 2, 4, 6, 8 and 24 hours, and the sampling times of the sustained release test were 0.5, 1, 2 , 4, 6, 8, 24, 48, 96, 168, 264 and 336 hours.

During each sampling, 4 ml of PBS was aspirated from the glass bottle, and then the same volume of fresh PBS was added, so that the total volume of PBS was maintained at 40 ml. The sampled PBS after being diluted was analyzed for its drug content by an ELISA reader, and the cumulative drug release amount of the film at each time point was confirmed by calculation.

In addition, in order to confirm the total drug content in each film, the film was cut into a plurality of small-sized films and placed in a glass sample bottle.

4 ml PBS was added to the glass bottle, and the glass bottle was ultrasonicated to extract the drug in the film, and the extraction time lasted about 30 minutes. After that, the drug concentration in PBS was analyzed by high-performance liquid chromatography (HPLC) to confirm the total drug amount contained in the film.

The cumulative drug release rate (%) of the film at each time point was calculated by the following formula.

Cumulative drug release rate % of the film at each time point=Cumulative drug release amount at each time point/Total drug amount contained in the film*100

2. Results

(1) Results of Immediate Release Test for Drug-Containing Multilayer Films M-01, M-02, M-03, M-04, M-05 and M-06

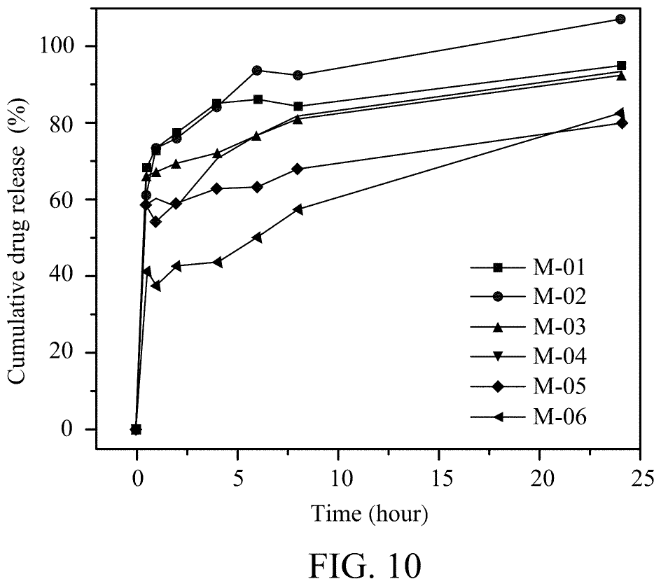

The results of the immediate release test for the drug-containing multilayer films M-01, M-02, M-03, M-04, M-05 and M-06 are shown in FIG. 10.

Based on FIG. 10, it is known that when the main component of the drug-containing layers in the drug-containing multilayer films were composed of low-viscosity polylactic acid (0.4-0.6 dl/g), regardless of whether the drug-containing layers contained polyethylene glycol (polyethylene glycol 300, polyethylene glycol 6000, or a combination thereof) or not, the drug release curves of the drug-containing multilayer films all showed an initial rapid release phenomenon, wherein 1-hour cumulative drug release rates all could reach more than 40%, and 24-hour cumulative drug release rates even could reach more than 80%.

The results mentioned above show that when low-viscosity polylactic acid (0.4-0.6 dl/g) is used as the main component of the drug-containing layer, the structure of the drug-containing layer is relatively loose, and thus the drug is easily released from the film through the diffusion mechanism. Therefore, when the drug-containing layer is composed of low-viscosity polylactic acid (0.4-0.6 dl/g), the drug-containing multilayer film therefrom is suitable as an immediate release film.

(2) Results of Immediate Release Testing for Drug-Containing Multilayer Films M-02, M-07, M-08, M-09 and M-10

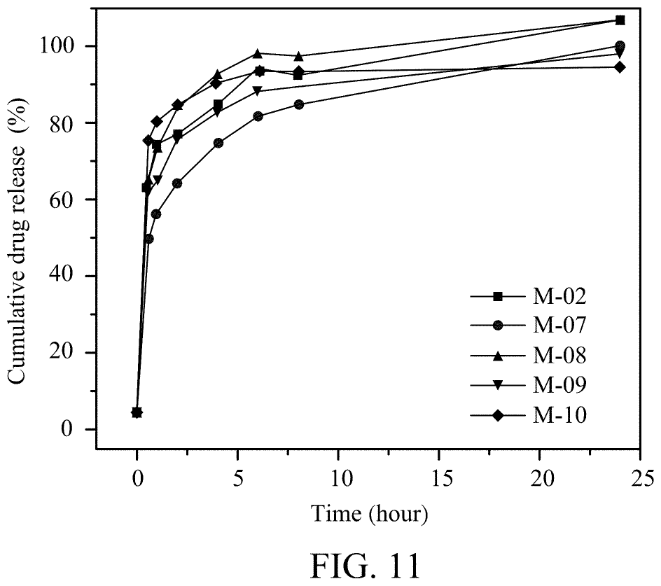

In order to confirm the effect of protective layers with different ingredients on drug release, the results of the immediate release test for the drug-containing multilayer film M-02 (without protective layer) and the drug-containing multilayer films M-07, M-08, M-09 and M-10 (with a protective layer) are shown in FIG. 11.

Based on FIG. 11, it is known that compared to the drug-free multilayer film M-02 without protective layer, the drug-containing multilayer film M-07 with a protective layer composed of low viscosity polylactic acid (0.4-0.6 dl/g) can reduce 0-hour to 24-hour cumulative drug release rates. When the component of the protective layer has polyethylene glycol 300 or polyethylene glycol 6000, the cumulative drug release rate of the drug-containing multilayer film does not show a decreasing trend, which indicates that the presence of polyethylene glycol makes the structure of the protective layer have porosity, and the effect of the physical barrier is not obvious.

(3) Results of Sustained Release Test for Drug-Containing Multilayer Films M-11 and M-12

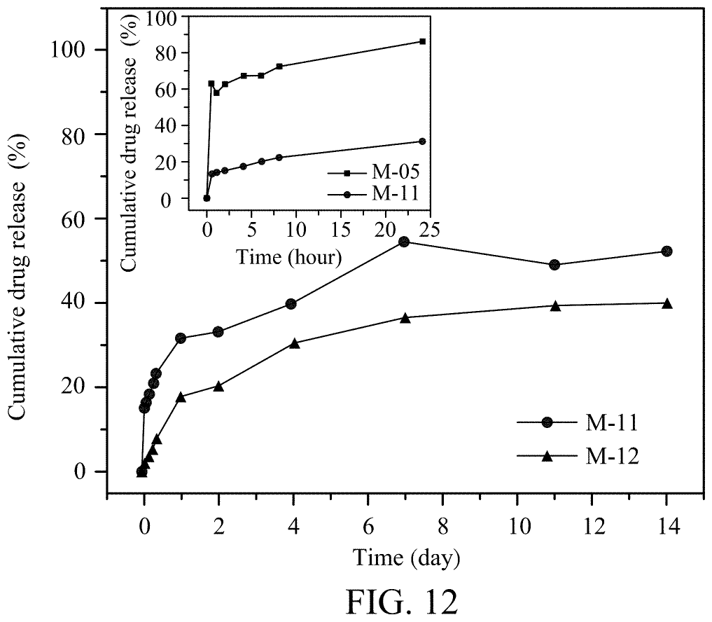

In order to confirm the effect of viscosity of polylactic acid and protective layer of the drug-containing multilayer film on the drug release of the drug-containing multilayer film, the results of the release test for the drug-containing multilayer film M-02 (without protective layer) and the drug-containing multilayer films M-11 and M-12 are shown in FIG. 12.

Based on FIG. 12, it is known that compared to the drug-containing multilayer film M-05 whose main component of the drug-containing layer is composed of low-viscosity polylactic acid (0.4-0.6 dl/g), the drug-containing multilayer film M-11 whose the main component of the drug-containing layer is composed of high-viscosity polylactic acid (1.6-2.4 dl/g) has a significant downward trend on the 0-1 hour cumulative drug release rate, and 24-hour cumulative drug release rate thereof has also decreased from 85% to 32%.

In addition, the drug-containing multilayer films (M-11 and M12) whose main components of drug-containing layers are composed of high-viscosity polylactic acid (1.6-2.4 dl/g) do not show a significant rapid release in the early stage, and with the extension of time, the 14-day cumulative drug release rate is only 50%, which means that the drug in this kind of drug-containing multilayer film can be sustainably released for more than 14 days.

This result shows that when the high viscosity polylactic acid (1.6-2.4 dl/g) is used as the main component of the drug-containing layer, the structure of the drug-containing layer is relatively compact, which can prolong the release time of the drug from the film.

Therefore, when the drug-containing layer is composed of high viscosity polylactic acid (1.6-2.4 dl/g), the drug-containing multilayer film therefrom is suitable as a sustained-release film.

In addition, the results of the drug-containing multilayer films M-11 and M12 also show that compared to the drug-containing multilayer film M-11 without protective layer, the drug-containing multilayer film M-12 with a protective layer has not shown initial rapid release (0-1 hour cumulative drug release rate), and its 14-day cumulative drug release rate is only about 40%. This shows that in the case where the main component of the drug-containing layer is composed of high-viscosity polylactic acid (1.6-2.4 dl/g), the presence of the protective layer can further reduce the cumulative drug release rate of the drug and prolong the drug release time.

(4) Results of Sustained Release Test for Drug-Containing Multilayer Films M-11, MS-1, MS-2 and MS-3

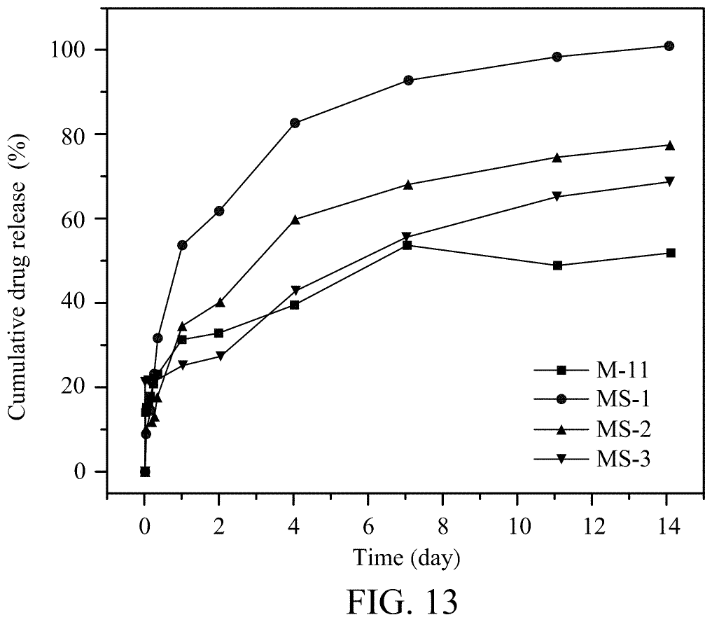

In order to confirm the effect of the microstructure of the drug-containing multilayer film on the drug release of the drug-containing multilayer film, the results of the release tests on the drug-containing multilayer film M-11 (the drug-containing layer without microstructure) and the drug-containing multilayer films MS-1, MS-2 and MS-3 are shown in FIG. 13.

When the number of microstructures of the drug-containing layer increases, the cumulative drug release rate will also increase accordingly. The cumulative drug release of the drug-containing multilayer film (MS-1) whose drug-containing layer has the largest number of microstructures per unit area reached 100% in 14 days, and when this is compared to the drug-containing multilayer film (M-11) without microstructure, the release rate is increased by 50%.

In contrast, when the number of microstructures per unit area of the drug-containing layer decreases, the degree of increase in the cumulative release rate of the drug also reduces. Compared to the drug-containing multilayer film M-11 without microstructure, the cumulative release rates of the drug-containing multilayer films MS-2 and MS-3 respectively increased by only 30% and 20% in 14 days.

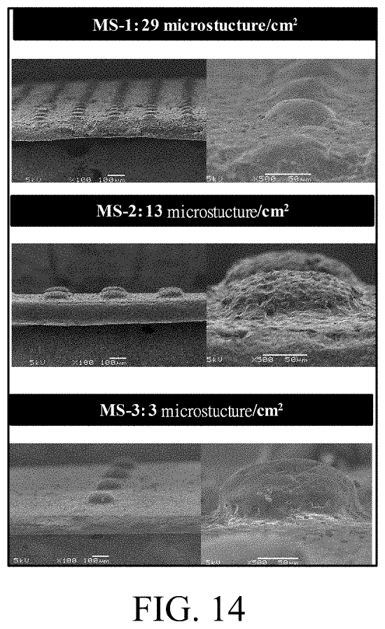

In addition, the results of scanning electron microscope analysis for the drug-containing multilayer films MS-1, MS-2 and MS-3 are shown in FIG. 14.

(5) Results of Sustained Release Test for Drug-Containing Multilayer Films MS-1, MS-2, MS-1-B and MS-2-B

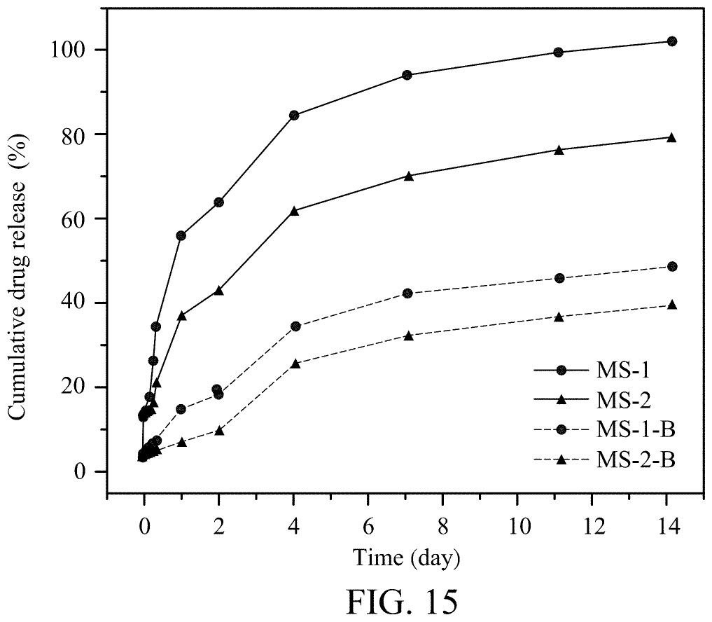

In order to confirm the effect of the protective layer on the drug release of a drug-containing multilayer film with a microstructured drug-containing layer, the results of the release test for the drug-containing multilayer films MS-1, MS-2, MS-1-B and MS-2-B are shown in FIG. 15.

According to FIG. 15, it known that compared to the drug-containing multilayer film MS-1, the drug-containing multilayer film MS-1-B with a protective layer has a cumulative release rate decreasing from 100% to 50% on the 14th day, and compared to the drug-containing multilayer film MS-2, the drug-containing multilayer film MS-2-B with a protective layer has a cumulative release rate decreasing from 80% to 40% on the 14th day.

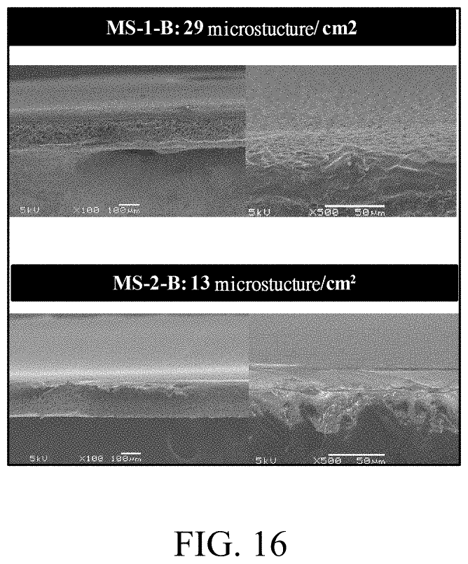

Also, the results of the scanning electron microscope analysis for the drug-containing multilayer films MS-1-B and MS-2-B are shown in FIG. 16.

According to FIG. 16, it known that the microstructures of the drug-containing layers of the drug-containing multilayer films MS-1-B and MS-2-B have been covered by the protective layers, so that the surface area was greatly reduced, and that resulted in the drug being less likely to diffuse out of the film, thereby reducing the cumulative release rate of the drug.

It will be apparent to those skilled in the art that various modifications and variations can be made to the disclosed embodiments. It is intended that the specification and examples be considered as exemplary only, with a true scope of the disclosure being indicated by the following claims and their equivalents.

1. A drug-containing multilayer film, comprising:
a drug-containing layer; and
an anti-adhesion layer over a surface of the drug-containing layer,
wherein the drug-containing layer is composed of a first composition which comprises a first polymer material and a drug, and the first polymer material comprises at least one selected from the group consisting of:
polylactic acid (PLA) and polyethylene glycol (PEG), and
a weight ratio of the first polymer material to the drug is 1:0.01-0.3, and
wherein the anti-adhesion layer is composed of a second composition which comprises a second polymer material, and the second polymer material comprises at least one selected from the group consisting of:
polylactic acid (PLA) and polyethylene glycol (PEG).

2. The drug-containing multilayer film as claimed in claim 1, wherein the first polymer material comprises the polylactic acid, and the polylactic acid is a first polylactic acid or a second polylactic acid, wherein viscosity of the first polylactic acid is 0.05-1 dl/g, and viscosity of the second polylactic acid is 1.1-3.0 dl/g.

3. The drug-containing multilayer film as claimed in claim 2, wherein the first polymer material is the polylactic acid, and the polylactic acid is the first polylactic acid, and the drug comprises:
an analgesic, an anti-inflammatory and analgesic drug or a combination thereof.

4. The drug-containing multilayer film as claimed in claim 2, wherein the first polymer material is the polylactic acid, and the polylactic acid is the second polylactic acid, and the drug comprises:
an analgesic, an antibiotic or a combination thereof.

5. The drug-containing multilayer film as claimed in claim 1, wherein the first polymer material comprises the polylactic acid and the polyethylene glycol, and the polylactic acid is a first polylactic acid or a second polylactic acid, and the polyethylene glycol is a first polyethylene glycol, a second polyethylene glycol or a combination thereof, wherein viscosity of the first polylactic acid is 0.05-1 dl/g, and viscosity of the second polylactic acid is 1.1-3.0 dl/g, and wherein an average molecular weight of the first polyethylene glycol is 100-1,000, and an average molecular weight of the first second polyethylene glycol is 4,000-10,000.

6. The drug-containing multilayer film as claimed in claim 5, wherein in the first polymer material, a weight ratio of the polylactic acid to the polyethylene glycol is 1:0.05-1.

7. The drug-containing multilayer film as claimed in claim 5, wherein the first polymer material is a combination of the polylactic acid and the polyethylene glycol, and the polylactic acid is the first polylactic acid or the second polylactic acid, and the polyethylene glycol is the first polyethylene glycol or the second polyethylene glycol.

8. The drug-containing multilayer film as claimed in claim 7, wherein the polylactic acid is the first polylactic acid, and the drug comprises:
an analgesic, an anti-inflammatory and analgesic drug or a combination thereof.

9. The drug-containing multilayer film as claimed in claim 7, wherein the polylactic acid is the second polylactic acid, and the drug comprises:
an analgesic, an antibiotic or a combination thereof.

10. The drug-containing multilayer film as claimed in claim 5, wherein the first polymer material is a combination of the polylactic acid and the polyethylene glycol, and the polylactic acid is the first polylactic acid or the second polylactic acid, and the polyethylene glycol is a combination of the first polyethylene glycol and the second polyethylene glycol, and wherein a weight ratio of the first polyethylene glycol to the second polyethylene glycol is 1:0.01-10.

11. The drug-containing multilayer film as claimed in claim 10, wherein the polylactic acid is the first polylactic acid, and the drug comprises:
an analgesic, an anti-inflammatory and analgesic drug or a combination thereof.

12. The drug-containing multilayer film as claimed in claim 10, wherein the polylactic acid is the second polylactic acid, and the drug comprises:
an analgesic, an antibiotic, or a combination thereof.

13. The drug-containing multilayer film as claimed in claim 1, wherein the drug-containing layer has a plurality of microstructures protruding from the other surface thereof.

14. The drug-containing multilayer film as claimed in claim 13, wherein the microstructure has an upper surface, and a shape of the upper surface comprises an arc-shaped surface or a flat surface.

15. The drug-containing multilayer film as claimed in claim 13, wherein a ratio of height to width of a bottom surface of the microstructure is 1:2-4.

16. The drug-containing multilayer film as claimed in claim 13, wherein the drug-containing layer has a plurality of microstructures.

17. The drug-containing multilayer film as claimed in claim 16, wherein density of the microstructures on the drug-containing layer is 0.1-100 microstructure/cm².

18. The drug-containing multilayer film as claimed in claim 1, wherein the second polymer material comprises the polylactic acid, and viscosity of the polylactic acid is 1.1-3.0 dl/g.

19. The drug-containing multilayer film as claimed in claim 1, wherein the second polymer material comprises the polylactic acid and the polyethylene glycol, and the polyethylene glycol is a third polyethylene glycol, a fourth polyethylene glycol or a combination thereof, wherein viscosity of the polylactic acid is 1.1-3.0 dl/g, and wherein an average molecular weight of the third polyethylene glycol is 100-1,000, and an average molecular weight of the fourth polyethylene glycol is 4,000-10,000.

20. The drug-containing multilayer film as claimed in claim 1, further comprising a protective layer over the other surface of the drug-containing layer, wherein the drug-containing layer is between the anti-adhesion layer and the protective layer, and
wherein the protective layer is composed of a third composition which comprises a third polymer material, and the third polymer material comprises at least one selected from the group consisting of:
polylactic acid and polyethylene glycol.

21. The drug-containing multilayer film as claimed in claim 20, wherein the third polymer material comprises the polylactic acid, and the polylactic acid is a third polylactic acid or a fourth polylactic acid, wherein viscosity of the third polylactic acid is 0.05-1 dl/g, and viscosity of the fourth polylactic acid is 1.1-3.0 dl/g.

22. The drug-containing multilayer film as claimed in claim 20, wherein the third polymer material comprises the polylactic acid and the polyethylene glycol, and the polylactic acid is a third polylactic acid or a fourth polylactic acid, and the polyethylene glycol is a fifth polyethylene glycol, a sixth polyethylene glycol or a combination thereof, wherein viscosity of the third polylactic acid is 0.05-1 dl/g, and viscosity of the fourth polylactic acid is 1.1-3.0 dl/g, and wherein an average molecular weight of the fifth polyethylene glycol is 100-1,000, and an average molecular weight of the sixth polyethylene glycol is 4,000-10,000.

23. The drug-containing multilayer film as claimed in claim 22, wherein the third polymer material is a combination of the polylactic acid and the polyethylene glycol, and the polylactic acid is the third polylactic acid or the fourth polylactic acid, and the polyethylene glycol is the fifth polyethylene glycol or the sixth polyethylene glycol.

24. The drug-containing multilayer film as claimed in claim 22, wherein the third polymer material is a combination of polylactic acid and polyethylene glycol, and the polylactic acid is the third polylactic acid or the fourth polylactic acid, and the polyethylene glycol is a combination of the fifth polyethylene glycol and the sixth polyethylene glycol, and wherein a weight ratio of the fifth polyethylene glycol to the sixth polyethylene glycol is 1:0.05-10.

25. A method for forming a drug-containing multilayer film, comprising the following manner (a) or (b):
the manner (a), comprising:
(i) drying a first solution to form a film to form a drug-containing layer; and
(ii) drying a second solution to form a film over a surface of the drug-containing layer to form an anti-adhesion layer;
the manner (b), comprising:
(i′) drying a second solution to form a film to form an anti-adhesion layer; and
(ii′) drying a first solution to form a film over the anti-adhesion layer to form a drug-containing layer,
wherein a solute of the first solution comprises a first polymer material and a drug, and the first polymer material comprises at least one selected from the group consisting of:
polylactic acid (PLA) and polyethylene glycol (PEG), and
a content of the first polymer material in the first solution is 5-30 wt %, and
a weight ratio of the first polymer material to the drug is 1:0.01-0.3, and
wherein a solute of the second solution comprises a second polymer material, and the second polymer material comprises at least one selected from the group consisting of:
polylactic acid and polyethylene glycol, and
a content of the second polymer material in the second solution is 5-30 wt %.

26. The method for forming a drug-containing multilayer film as claimed in claim 25, wherein the first polymer material comprises the polylactic acid, and the polylactic acid is a first polylactic acid or a second polylactic acid, wherein viscosity of the first polylactic acid is 0.05-1 dl/g, and viscosity of the second polylactic acid is 1.1-3.0 dl/g.

27. The method for forming a drug-containing multilayer film as claimed in claim 25, wherein the first polymer material comprises the polylactic acid and the polyethylene glycol, and the polylactic acid is a first polylactic acid or a second polylactic acid, and the polyethylene glycol is a first polyethylene glycol, a second polyethylene glycol or a combination thereof, wherein viscosity of the first polylactic acid is 0.05-1 dl/g, and viscosity of the second polylactic acid is 1.1-3.0 dl/g, and wherein an average molecular weight of the first polyethylene glycol is 100-1,000, and an average molecular weight of the second polyethylene glycol is 4,000-10,000.

28. The method for forming a drug-containing multilayer film as claimed in claim 25, wherein in the manner (a), a method for forming the drug-containing layer comprises:
pouring the first solution on a plate with at least one recess, and drying it to form a film on the plate with at least one recess to make the drug-containing layer with at least one microstructure protrude from the other surface thereof.

29. The method for forming a drug-containing multilayer film as claimed in claim 28, wherein the recess has an upper surface, and a shape of the upper surface comprises an arc-shaped surface or a flat surface.

30. The method for forming a drug-containing multilayer film as claimed in claim 25, wherein the second polymer material comprises the polylactic acid, and viscosity of the polylactic acid is about 1.1-3.0 dl/g.

31. The method for forming a drug-containing multilayer film as claimed in claim 25, wherein the second polymer material comprises the polylactic acid and the polyethylene glycol, and the polyethylene glycol is a third polyethylene glycol, a fourth polyethylene glycol or a combination thereof, wherein viscosity of the polylactic acid is 0.05-1 dl/g, and wherein an average molecular weight of the third polyethylene glycol is about 100-1,000, and an average molecular weight of the fourth polyethylene glycol is about 4,000-10,000.

32. The method for forming a drug-containing multilayer film as claimed in claim 25, wherein
the manners (a) and (b) further comprise:
(iii) drying a third solution to form a film over the drug-containing layer to form a protective layer, wherein the drug-containing layer is between the anti-adhesion layer and the protective layer, and
wherein a solute of the third solution comprises a third polymer material, and the third polymer material comprises at least one selected from the group consisting of:
polylactic acid and polyethylene glycol, and
a content of the third polymer material in the third solution material is about 5-30 wt %.
